(19) United States
(12) Patent Application Publication
(10) Pub. No.: US 2010/0076217 A1
(43) Pub. Date: Mar. 25, 2010

(54) ACYLOXYLATION CATALYST AND PROCESS FOR ITS PRODUCTION

(75) Inventors: Yoshimi Yamamoto, Minato-ku (JP); Yumiko Watanabe, Minato-ku (JP); Seiji Sato, Minato-ku (JP)

Correspondence Address: SUGHRUE MION, PLLC, 2100 PENNSYLVANIA AVENUE, N.W., SUITE 800, WASHINGTON, DC, 20037, US

(73) Assignee: SHOWA DENKO K.K., MINATO-KU TOKYO (JP)

(21) Appl. No.: 12/513,620

(22) PCT Filed: Dec. 5, 2007
(86) PCT No.: PCT/JP2007/073904

§ 371 Date: May 5, 2009

(30) Foreign Application Priority Data
Dec. 20, 2006 (JP) 2006-324897

(51) Int. Cl. C07C 69/02; B01J 23/44; B01J 23/745; B01J 23/52

(52) U.S. Cl. 560/231; 502/326; 502/330

(57) ABSTRACT

An acyloxylation catalyst is obtained by loading (a) a first component containing at least one element of Groups 8, 9, 10 and 11 of the Periodic Table, (b) a second component containing an element which is at least one element of Groups 8, 9, 10 and 11 of the Periodic Table and which is different from the element of the first component, and (c) a third component containing an element which is a component that produces a precipitation-starting pH below the precipitation-starting pH of the first component and second component and which is different from the elements of the first component and second component, onto (d) a support. A catalyst is obtained that can be used to efficiently carry out acyloxylation for economical production of acyloxylated compounds.

### TECHNICAL FIELD

The present invention relates to an acyloxylation catalyst production process, to an acyloxylation catalyst obtained by it and to an acyloxylation process using the catalyst.

Acyloxylation is a synthetic reaction utilized for a variety of useful compounds used as flavorings, medicines and agricultural chemicals, organic synthetic intermediates and polymerizable materials.

### BACKGROUND ART

The prior art includes the knowledge of acyloxylation using compounds having hydrogen at a benzyl position such as toluene, xylene or the like or compounds having hydrogen at an allyl position such as propylene, cyclohexene or the like, with oxygen and carboxylic acids such as acetic acid.

The catalysts that have been developed for such acyloxylation are homogeneous catalysts such as palladium acetate and non-heterogeneous catalysts comprising palladium supported on a support such as silica.

Japanese Unexamined Patent Publication No. 2001-269577 discloses a process for economical production of acyloxy compounds using a catalyst comprising a metal of Groups 8-11 of the Periodic Table such as palladium, and an alkali metal such as sodium or potassium and/or a metal of Groups 12-16 of the Periodic Table such as antimony, bismuth or tellurium, on a support such as active carbon.

Japanese Patent Public Inspection No. 2001-521817 discloses a catalyst comprising palladium and gold, with a third metal such as magnesium, calcium, barium, zirconium or cerium as their oxides or as a mixture of their oxides and their metallic forms on a support, as well as a process whereby the support is impregnated with a solution of water-soluble salts of the palladium, gold and third metal and subsequently reacted with an alkaline compound to fix it as a water-insoluble compound, and the fixed palladium and gold are then reduced to their metallic states while the third metal is reduced to its oxide or a mixture of its oxide and metallic form.

Also, Japanese Unexamined Patent Publication No. 2005-296858 discloses a catalyst comprising an amphoteric metal such as palladium, gold or zinc and an alkali metal, reporting that addition of zinc can yield a precious metal surface area equivalent to a gas phase reduction process even when employing a liquid phase reduction process.

However, these conventional acyloxylation catalysts have had drawbacks such as insufficient balance of performance between initial reaction activity, selectivity and sustained activity.

### DISCLOSURE OF THE INVENTION

It is an object of the present invention, which has been accomplished in light of the circumstances described above, to overcome the aforementioned problems by providing a catalyst for economical production of acyloxy compounds, as well as a process for its production.

As a result of much diligent research directed toward solving the problems mentioned above, the present inventors have discovered that by loading onto a support at least a first component containing an element of Group 8, 9, 10 or 11 of the Periodic Table, a second component containing an element which is different from the element of the first component, and a third component containing an element that is different from the elements of the first component and second component, it is possible to obtain a catalyst that can accomplish acyloxylation efficiently to allow economical production of acyloxylated products, and the invention has been completed upon this discovery.

The invention therefore provides the following (1)-(14).

(1) A process for production of an acyloxylation catalyst comprising a step of loading (a) a first component containing at least one element of Groups 8, 9, 10 and 11 of the Periodic Table, (b) a second component containing an element which is at least one element of Groups 8, 9, 10 and 11 of the Periodic Table and which is different from the element of the first component and (c) a third component containing an element which is a component that has a precipitation-starting pH below the precipitation-starting pH of the first component and second component and which is different from the elements of the first component and second component, together, onto (d) a support.

(2) A process for production of an acyloxylation catalyst according to (1) above, wherein the step of loading onto the (d) support is followed by non-solubilizing treatment.

(3) A process for production of an acyloxylation catalyst according to (1) or (2) above, wherein the step of loading onto the (a) support is followed by reduction treatment.

(4) A process for production of an acyloxylation catalyst according to (3) above, wherein the reduction treatment is followed by contact with an acid and/or chelate.

(5) A process for production of an acyloxylation catalyst according to any one of (1)-(4) above, comprising further (e) adding a fourth component containing at least one element of Groups 1 and 2 of the Periodic Table except for hydrogen.

(6) A process for production of an acyloxylation catalyst according to any one of (1)-(5) above, wherein the (a) first component includes palladium.

(7) A process for production of an acyloxylation catalyst according to any one of (1)-(6) above, wherein the (b) second component includes an element of Group 11 of the Periodic Table.

(8) A process for production of an acyloxylation catalyst according to (7) above, wherein the (b) second component includes at least one element selected from the group consisting of gold and copper.

(9) A process for production of an acyloxylation catalyst according to any one of (1)-(8) above, wherein the (c) third component includes an element of Groups 3-13 of the Periodic Table.

(10) A process for production of an acyloxylation catalyst according to any one of (1)-(9) above, wherein the proportion of the loading weight of the element of the (b) second component to the loading weight of the element of the (a) first component is 0.4-1.5.

(11) A process for production of an acyloxylation catalyst according to any one of (1)-(10) above, wherein the proportion of the loading weight of the element of the (c) third component to the loading weight of the element of the (b) second component is 0.1-0.5.

(12) An acyloxylation catalyst obtained by a process according to any one of (1)-(11) above.

(13) A process for production of acyloxy compounds, comprising reacting, in the presence of a catalyst obtained by a process according to any one of (1)-(11) above, a compound represented by the general formula (1): CHR¹R²—X (wherein R¹ and R² each independently represent hydrogen or an organic residue and X represents an optionally substituted aromatic hydrocarbon residue or optionally substituted olefin residue) or ethylene, with a carboxylic acid represented by the general formula (2): R³—COOH (wherein R³ represents hydrogen or an organic residue) and oxygen to produce a compound represented by the general formula (3): R³—COO—CR¹R²—X (wherein R¹, R², R³ and X have the same definitions as above) or vinyl acetate.

(14) A process for production of an acyloxy compound according to (13) above, wherein the reaction is conducted also in the presence of at least one compound selected from the group consisting of basic compounds, nitrogen-containing compounds and phosphorus-containing compounds.

According to the invention it is possible to obtain an acyloxylation catalyst with an excellent balance of performance between initial reaction activity, selectivity and sustained activity, and thus produce acyloxy compounds efficiently and economically.

### BRIEF DESCRIPTION OF THE DRAWINGS

FIG. 1 is a graph illustrating the method of measuring the precipitation-starting pH as specified according to the invention.

### BEST MODE FOR CARRYING OUT THE INVENTION

Preferred modes of the invention will now be explained in detail with the understanding that the invention is not limited only to these modes, and that various modifications may be implemented such as are within the spirit and scope of the invention.

The acyloxylation catalyst of the invention is an acyloxylation catalyst comprising an active substance supported on a support.

The (a) first component and (b) second component for the invention are elements of Groups 8-11 of the Periodic Table according to the IUPAC Revised Nomenclature of Inorganic Chemistry (1989), and as examples there may be mentioned iron, ruthenium, osmium, cobalt, rhodium, iridium, nickel, palladium, platinum, copper, silver and gold. Of these, ruthenium, rhodium, palladium and silver are preferred as the element of the (a) first component, with palladium being more preferred, and osmium, iridium, platinum, copper and gold are preferred as the element of the (b) second component, with gold being more preferred. The (a) first component includes the element as the main essential catalyst for the reaction, while the (b) second component includes an element as a co-catalyst to increase the reaction efficiency. The elements of the (a) first component and (b) second component may be one of each or appropriate combinations of two or more elements.

As examples of palladium compounds for the (a) first component and (b) second component there may, be mentioned metallic palladium, ammonium hexachloropalladate, potassium hexachloropalladate, sodium hexachloropalladate, ammonium tetrachloropalladate, potassium tetrachloropalladate, sodium tetrachloropalladate, potassium tetrabromopalladate, palladium oxide, palladium chloride, palladium bromide, palladium iodide, palladium nitrate, palladium sulfate, palladium acetate, potassium palladate dinitrosulfite, chlorocarbonylpalladium, dinitrodiaminepalladium, tetraminepalladium chloride, tetraminepalladium nitrate, tetraminepalladium hydroxide, cis-dichlorodiaminepalladium, trans-dichlorodiaminepalladium, dichloro(ethylenediamine)palladium, tetracyanopotassium palladate and the like.

The loading weights of the (a) first component and (b) second component are preferably 0.01-20 mass % and more preferably 0.1-10 mass % with respect to the final catalyst. The loading weight can be determined by dissolving the final catalyst in an acid and performing ICP analysis to determine the content of the element of interest.

The (a) first component, (b) second component and (c) third component may be each loaded by, for example, impregnation as an aqueous solution into the (d) support. In this case, the (a) first component, (b) second component and (c) third component are preferably added together to a single aqueous solution and the aqueous solution impregnated into the (d) support for loading.

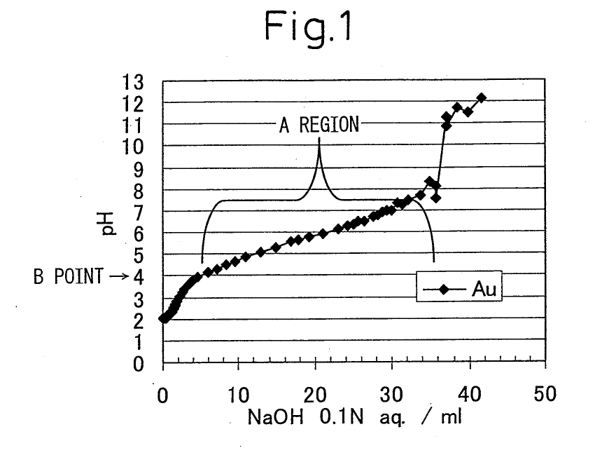

The precipitation-starting pH according to the invention can be determined by the following method. Specifically, after each metal or metal compound is dissolved in an acidic aqueous solution and an alkaline aqueous solution is added dropwise to raise the pH, the increase in pH decelerates in a given region and the precipitation-starting pH is defined as the pH at the start of deceleration. For example, by measuring the pH of an aqueous solution of chloroauric acid while adding a sodium hydroxide aqueous solution dropwise and plotting the pH against the amount of dropwise addition, a titration curve such as shown in FIG. 1 is obtained. When a given amount has been added, a relatively stable state is observed in which the pH no longer increases significantly even with addition of the sodium hydroxide aqueous solution, as in A region shown in the drawing. The pH at the start point (B point) of this stable state is the precipitation-starting pH. For the measurement, it is important that the titration is started with the metals and metal compounds dissolved to form a homogeneous aqueous solution before precipitation begins.

The chemical species, concentration and amount of addition are not particularly restricted so long as the alkaline aqueous solution used for the titration is an alkaline aqueous solution that allows pH changes in acidic aqueous solutions to be observed. For comparative measurement in series, however, titration must be performed on each acidic aqueous solution using the same alkaline aqueous solution. The titration may be performed with a sodium hydroxide aqueous solution, for example.

The component selected as the (c) third component may be one with a precipitation-starting pH below the precipitation-starting pH of the (a) first component and (b) second component, based on the precipitation-starting pH measured in the manner described above, and containing an element different from the elements of the first component and second component. As third components with a lower precipitation-starting pH than a first component (a) containing palladium, for example, there may be mentioned compounds containing scandium, titanium, molybdenum, tungsten or iron, or these elements alone. The compounds may be oxides, hydroxides, halides oxyhalides, alkoxides, aliphatic carboxylates such as acetates, nitrates, carbonates, phosphates, borates and the like, as well as their hydrates. Using iron as an example, there may be used metallic iron, iron hydroxide, iron chloride, iron bromide, iron iodide, iron perchlorate, iron methoxide, iron ethoxide, iron acetate, iron propionate, iron acrylate, iron nitrate, iron carbonate, iron sulfate, iron phosphate, iron acetate, iron citrate, iron gluconate, iron fumarate, iron oxalate, iron borate and the like, or hydrates of the foregoing. Preferred as the (c) third component among the above are compounds containing at least one element selected from scandium, titanium and iron or at least one of these elements. The (c) third component may be a single type of species or an appropriate combination of two or more elements.

It is presumed that addition of the (c) third component selected as explained above promotes coexistence or homogeneous mixture of the (a) first component and (b) second component, producing an effect of higher activity and selectivity and minimal activity reduction.

The loading weight of the element of the (c) third component for the catalyst of the invention is preferably 0.001-20 mass % and more preferably 0.01-10 mass %. The loading weight can be determined by dissolving the final catalyst in an acid and then performing ICP analysis of the content of the element to be measured, in the same manner as for the element of the (a) first component or (b) second component.

The ratio of the loading weight of the element of the (b) second component with respect to the loading weight of the element of the (a) first component is preferably 0.4-1.5 and more preferably 0.8-1.2. The ratio of the loading weight of the element of the (c) third component with respect to the loading weight of the element of the (b) second component is preferably 0.1-0.5 and more preferably 0.2-0.4.

An acid washing step may also be carried out in the catalyst production process of the invention, and because the loading weight will decrease as a result, the loading weight is defined as the value prior to such acid washing.

The (d) support for the catalyst of the invention may be, for example, a metal oxide such as silica, alumina, zirconia or titania, or carbon, charcoal, active carbon, asbestos, silica-alumina, zeolite, an organosol-gel, an ion exchange resin, clay, a carbonate, or the like. Preferred among these are metal oxides such as silica, alumina, zirconia or titania, as well as active carbon and zeolite.

There are no particular restrictions on the area-to-weight ratio of the support, but from the standpoint of balance between dispersion of the catalyst components and mechanical strength of the support, the value measured by B.E.T. is preferably 10-1500 m²/g and more preferably 100-500 m²/g.

The starting material for active carbon may be wood, xylogen, coconut shell, husks, an organic polymer or the like. Xylogen and coconut shell are preferred among the above. The area-to-weight ratio of active carbon used as the support is preferably in the range of 800-2500 m²/g and especially in the range of 1000-1800 m²/g, from the standpoint of balance between dispersion of the catalyst components and mechanical strength of the support.

The form of the support may be, for example, powdered, crushed, granular or columnar, which may be selected as appropriate for the reaction system to be used, such as a fixed bed system, fluidized bed system, suspended catalyst system or the like.

The (a) first component, (b) second component and (c) third component are preferably subjected to non-solubilizing treatment for precipitation onto the (d) support.

Non-solubilizing treatment is a process in which a catalyst precursor, obtained by impregnating the (d) support with an aqueous solution of the (a) first component, (b) second component and (c) third component, is contacted with an acidic or alkaline aqueous solution to precipitate the (a) first component, (b) second component and (c) third component. The catalyst precursor referred to here is an intermediate catalyst between the point where the substances to be loaded are contacted with or loaded on the support, and completion of the various steps to obtain the final catalyst. If the aqueous solution comprising the (a) first component, (b) second component and (c) third component is acidic it is contacted with an alkaline aqueous solution, and if it is alkaline it is contacted with an acidic aqueous solution. It will be contacted with an alkaline aqueous solution in most cases where the invention is implemented.

The acid to be used for an acidic aqueous solution is not particularly restricted, and as examples there may be mentioned inorganic acids such as hydrochloric acid, sulfuric acid, nitric acid and heteropolyacids, and organic acids such as acetic acid, phosphoric acid, oxalic acid, citric acid and gluconic acid.

As examples of alkaline aqueous solutions there may be mentioned aqueous solutions of alkaline compounds such as alkali metal or alkaline earth metal hydroxides, alkali metal or alkaline earth metal bicarbonates, alkali metal or alkaline earth metal carbonates and alkali metal or alkaline earth metal silicates. Preferred alkali metals include lithium, sodium and potassium. Preferred alkaline earth metals include barium and magnesium. Most preferred for use are sodium metasilicate, potassium metasilicate, sodium hydroxide, potassium hydroxide and barium hydroxide.

The method of non-solubilizing treatment may be, for example, a method in which the catalyst precursor is immersed in an acidic aqueous solution or alkaline aqueous solution, or an acidic aqueous solution or alkaline aqueous solution is added dropwise to the catalyst precursor, for contact between them.

There are no particular restrictions on the amount of acidic aqueous solution or alkaline aqueous solution used in the non-solubilizing treatment or on the concentration of the aqueous solution. However, it is preferably adjusted for a post-treatment liquid phase pH in the range of 7-11.

The contact time between the acidic aqueous solution or alkaline aqueous solution used for non-solubilizing treatment is preferably 0.5-100 hours and especially 3-50 hours. A time of shorter than 0.5 hour may not allow sufficient non-solubilization to be accomplished. A time of longer than 100 hours may lead to damage to the (d) support depending on the type of washing solution and the type of support, and may promote re-dissolution of the (a) first component and (b) second component.

The contact temperature is preferably 10-80° C. and especially 20-60° C. A contact temperature of lower than 10° C. is not preferred because it may delay the reaction and prolong the treatment time. A contact temperature of higher than 80° C. is also not preferred because it may promote aggregation of the (a) first component and (b) second component.

According to the invention, reduction treatment is preferably carried out after the step of loading the (a) first component, (b) second component and (c) third component or after the non-solubilizing treatment step, in order to prevent elution of the (a) first component and (b) second component, in particular.

For example, liquid phase reduction may be conducted in a non-aqueous system using an alcohol or hydrocarbon, or an aqueous system. The reducing agent used may be a carboxylic acid or its salt, an aldehyde, hydrogen peroxide, or a saccharide, diborane, amine, hydrazine or the like. Specifically, there may be mentioned oxalic acid, potassium oxalate, formic acid, potassium formate, ammonium citrate, glucose, polyhydric phenols, hydrazine, formaldehyde, acetaldehyde, hydroquinone, sodium borohydride, potassium citrate and the like. Hydrazine is especially preferred.

For gas phase reduction, hydrogen, carbon monoxide, an alcohol, an aldehyde or an olefin such as ethylene, propene or isobutene is used as the reduction gas. Hydrogen is preferred for use. An inert gas may also be used as a diluent for gas phase reduction. Examples of inert gases include helium, argon, nitrogen and the like.

Removal of the (c) third component may improve the catalyst performance in some cases. Removal is preferably accomplished by washing after reduction treatment, for example. Washing removal of the (c) third component may be carried out by, for example, contacting it with an acid or chelating agent for its dissolution and removal.

As examples of such an acid there may be mentioned inorganic acids such as hydrochloric acid, sulfuric acid, nitric acid and heteropolyacid, and organic acids such as acetic acid, phosphoric acid, oxalic acid, citric acid and gluconic acid.

Washing removal of the (c) third component may also be accomplished by one of the following chelating agents instead of an acid. Various different treatments including acid treatment may also be carried out in combination.

A chelating agent is a compound with an electron donor (ligand) capable of coordination bonding with metal ions, and it is a substance that forms a metal complex or metal chelate compound by reacting with a metal ion.

As useful chelating agents there may be mentioned nitrilotriacetic acid, diethylenetriaminepentaacetic acid, hydroxyethylethylenediaminetriacetic acid, triethylenetetraaminehexaacetic acid, 1,3-propanediaminetetraacetic acid, 1,3-diamino-2-hydroxypropanetetraacetic acid, hydroxyliminodiacetic acid, dihydroxylglycine, glycoletherdiaminetetraacetic acid, L-glutamic diacetic acid and the like.

For washing, the chelating agent is dissolved in water and used as an aqueous solution. Dissolution can be easily achieved in most cases in a sodium hydroxide aqueous solution or an aqueous alkali solution such as ammonia water, but an organic solvent such as an alcohol may be used instead.

The washing removal method for the (c) third component is not particularly restricted and may involve immersion of the catalyst precursor in a washing solution. The immersion time is preferably 0.5-100 hours and especially 3-50 hours. A time of shorter than 0.5 hour may not allow sufficient washing to be accomplished. A time of longer than 100 hours is not preferred because it may lead to damage to the (d) support depending on the type of washing solution and the type of support, and may promote re-dissolution of the (a) first component and (b) second component.

The contact temperature is not particularly restricted but is preferably 10-80° C. and more preferably 20-60° C. A contact temperature of lower than 10° C. is not preferred because the reaction may be delayed and the treatment time prolonged. A contact temperature of higher than 80° C. is also not preferred because it may promote aggregation of the (a) first component and (b) second component.

It is also preferred to further add (e) a fourth component containing an element of Group 1 and/or Group 2 of the Periodic Table except for hydrogen to the catalyst. The addition may be accomplished by loading during production of the catalyst, or the component may be added to the reaction system after catalyst production and before use in reaction, or during reaction.

Loading the (e) fourth component onto the acyloxylation catalyst of the invention or adding it to the reaction system either before or after reaction may improve the conversion rate and selectivity for acyloxylation, and allow more economical production of acyloxylated products.

As examples of elements of Group 1 and/or Group 2 of the Periodic Table for the (e) fourth component there may be mentioned lithium, sodium, potassium, rubidium, cesium, francium, beryllium, magnesium, calcium, strontium and barium. Preferred among these are lithium, sodium, potassium and cesium. The metal element may be a single type of species or an appropriate combination of two or more elements.

The starting material to produce the (e) fourth component may be, specifically, a metal, oxide, hydroxide, halide, oxyhalide, alkoxide, acetate or other aliphatic carboxylate, nitrate, carbonate, phosphate or borate of an element of Group 1 or Group 2 of the Periodic Table. Using potassium as an example, it may be potassium metal, potassium hydroxide, potassium chloride, potassium bromide, potassium iodide, potassium sulfide, potassium acetate, potassium nitrate, potassium benzoate, potassium carbonate, potassium phosphate, potassium borate or the like.

The loading weight of the element in the (e) fourth component is preferably 0.001-40 mass % and more preferably 0.01-10 mass % with respect to the final catalyst. The loading weight can be determined by dissolving the final catalyst in an acid and then performing ICP analysis of the content of the element to be measured, in the same manner as for the element of the (a) first component or (b) second component. This loading weight range is preferred from the viewpoint of conversion rate and selectivity, as well as from the viewpoint of economy.

Loading of the (e) fourth component may be accomplished, for example, by impregnation, ion exchange, co-precipitation, deposition or kneading, but it is preferable to load it by impregnating the support with an aqueous solution containing the (e) fourth component.

The catalyst of the invention is preferably used for acyloxylation of a compound having hydrogen at the benzyl position such as toluene, xylene or the like, or a compound having hydrogen at the vinyl or allyl position such as ethylene, propylene, cyclohexene or the like, with a carboxylic acid such as acetic acid or with oxygen. That is, acyloxylation according to the invention is a reaction wherein a compound represented by general formula (3) above or vinyl acetate is produced by reaction between a compound represented by general formula (1) above or ethylene with a carboxylic acid represented by general formula (2) above and oxygen.

In the compound represented by general formula (1) used as a reaction starting material, the organic residues represented by R¹, R² in the formula may be, for example, C1-18 straight-chain, branched or cyclic saturated and/or unsaturated alkyl, C1-8 hydroxyalkyl, C2-20 alkoxyalkyl, C1-8 halogenated (for example, chlorinated, brominated or fluorinated) alkyl or aryl groups. Preferred among these are C1-10 saturated and/or unsaturated alkyl groups.

Examples of optionally substituted aromatic hydrocarbon residues represented by X include C1-18 straight-chain, branched or cyclic alkyl, C1-8 hydroxyalkyl, C2-20 alkoxy, optionally substituted phenoxy, C1-8 halogenated (for example, chlorinated, brominated or fluorinated) alkyl and hydroxyl groups, and phenyl groups optionally substituted with halogen atoms such as fluorine, chlorine, bromine and iodine.

As optionally substituted olefin residues represented by X there may be mentioned groups represented by the general formula (4): —CR⁴═CHR⁵ (wherein R⁴ and R⁵ represent hydrogen or organic residues). Examples of organic residues represented by R⁴ and R⁵ include C1-18 straight-chain, branched or cyclic saturated and/or unsaturated alkyl, C1-8 hydroxyalkyl, C2-20 alkoxyalkyl, C1-8 halogenated (for example, chlorinated, brominated or fluorinated) alkyl, aldehyde, aliphatic and/or aromatic ketone, carboxylic acid and aliphatic and/or aromatic carboxylic acid ester groups. Preferred for use among these are C1-10 straight-chain, branched or cyclic saturated and/or unsaturated alkyl, carboxylic acid and aliphatic and/or aromatic carboxylic acid ester groups. A ring may also be formed by R¹ or R² and R⁴ or R⁵.

As representative examples of compounds represented by general formula (1) there may be mentioned methacrylic acid, methyl methacrylate, ethyl methacrylate, propyl methacrylate, butyl methacrylate, 2-hydroxyethyl methacrylate and 2-ethylhexyl methacrylate, as well as ethylene, propylene, butene, pentene, hexene, heptene, nonene, decene, butadiene, cyclopentene, cyclopentadiene, cyclohexene, cyclohexadiene, cycloheptene, cyclooctene, cyclononene, cyclodecene, toluene, ethylbenzene, propylbenzene, butylbenzene, styrene, xylene, trimethylbenzene, tetramethylbenzene, pentamethylbenzene, hexamethylbenzene, methylbiphenyl, dimethylbiphenyl, diphenylmethane, triphenylmethane, methylphenol, methoxytoluene, ethoxytoluene, phenoxytoluene, and the like. Such compounds that include isomers may be any of the isomers alone and/or isomer mixtures. Preferred for use among those mentioned above are methyl methacrylate, butyl methacrylate, ethylene, propylene, butene, pentene, hexene, butadiene, cyclopentene, cyclopentadiene, cyclohexene, cyclohexadiene, toluene, xylene, trimethylbenzene, methoxytoluene and phenoxytoluene.

The carboxylic acid represented by general formula (2) used as a starting material in the acyloxy compound production process of the invention is not particularly restricted so long as it is a compound wherein R³ in the formula is hydrogen or an organic residue.

As organic residues represented by R³ there may be mentioned C1-18 straight-chain, branched or cyclic saturated and/or unsaturated alkyl, C1-8 hydroxyalkyl, C2-20 alkoxyalkyl, C2-20 acetoxyalkyl, C1-8 halogenated (for example, chlorinated, brominated or fluorinated) alkyl and optionally substituted aromatic groups. Preferred among these are C1-5 saturated and/or unsaturated alkyl groups.

As representative examples of carboxylic acids represented by general formula (2) there may be mentioned formic acid, acetic acid, propionic acid, butyric acid, valeric acid, caproic acid, caprylic acid, lauric acid, myristic acid, palmitic acid, stearic acid, acetoacetic acid, hydroxypropionic acid, isobutanoic acid, hydroxyisobutanoic acid, t-butylacetic acid, benzoic acid, acrylic acid and methacrylic acid. Of these, acetic acid, propionic acid, butyric acid, benzoic acid, acrylic acid and methacrylic acid are preferred, and acetic acid, propionic acid, acrylic acid and methacrylic acid are most preferred.

As examples of compounds represented by general formula (3) there may be mentioned vinyl acetate, vinyl acrylate, vinyl methacrylate, vinyl propionate, allyl acetate, allyl acrylate, allyl methacrylate, allyl propionate, benzyl acetate, benzyl acrylate, benzyl methacrylate, benzyl propionate, 4-methylbenzyl acetate, 4-methylbenzyl acrylate, 4-methylbenzyl methacrylate, 4-methylbenzyl propionate, cyclohexene acetate, cyclohexene acrylate, cyclohexene methacrylate, cyclohexene propionate, methyl α-acetoxymethyl acrylate, 1,4-xylene monoacetate and 1,4-xylene diacetate.

The molar ratio of the compound represented by general formula (1) and the carboxylic acid represented by general formula (2) at the start of the reaction may be in the range of, for example, 10/1-1/10. Even within the range specified above, it is more preferably 6/1-1/6. Addition of either or both the compound represented by general formula (1) and the carboxylic acid represented by general formula (2) in excess of the range specified above will not provide an effect of improved yield or shortened reaction time, and instead will lengthen the step of recovering excess starting materials, thus creating an economical disadvantage.

The oxygen used for the reaction may be atomic and/or molecular oxygen, but is preferably molecular oxygen. Molecular oxygen is preferably used as a mixture with an inert gas such as nitrogen, argon, helium or carbon dioxide. The oxygen concentration is more preferably adjusted to within a range that does not create a combustible composition for the gas in the reaction system.

Molecular oxygen and a gas mixture containing molecular oxygen may be supplied to either or both the liquid phase and/or gas phase of the reaction system. When molecular oxygen and a gas mixture containing molecular oxygen are supplied to the reaction system, the supply may be such as to produce an oxygen partial pressure in the range of 0.01-20 MPa.

The acyloxy compound production process of the invention accomplishes the aforementioned acyloxylation in the presence of an acyloxylation catalyst of the invention as described above.

The amount of catalyst used will depend on the types and combination of the compound represented by general formula (1) and the carboxylic acid represented by general formula (2), but generally it may be an amount so that the amount of the (a) first component is in the range of 0.01 mmol to 100 mol with respect to 1 mol of the compound represented by general formula (1). Using the catalyst within this range is preferred from the viewpoint of yield and economy.

From the viewpoint of conversion rate and selectivity, the process for production of acyloxy compounds according to invention is preferably carried out in a reaction system containing at least one compound selected from the group consisting of basic compounds, nitrogen-containing compounds and phosphorus-containing compounds. As examples of basic compounds there may be mentioned carboxylic acid salts of alkali metals and/or alkaline earth metals such as lithium formate, sodium formate, potassium formate, magnesium formate, calcium formate, barium formate, lithium acetate, sodium acetate, potassium acetate, magnesium acetate, calcium acetate, barium acetate, lithium propionate, sodium propionate, potassium propionate, magnesium propionate, calcium propionate, barium propionate, lithium acrylate, sodium acrylate, potassium acrylate, magnesium acrylate, calcium acrylate, barium acrylate, lithium methacrylate, sodium methacrylate, potassium methacrylate, magnesium methacrylate, calcium methacrylate, barium methacrylate, lithium benzoate, sodium benzoate, potassium benzoate, magnesium benzoate, calcium benzoate and barium benzoate; hydroxides of alkali metals and/or alkaline earth metals such as lithium hydroxide, sodium hydroxide, potassium hydroxide, magnesium hydroxide, calcium hydroxide and barium hydroxide; carbonic acid salts of alkali metals and/or alkaline earth metals such as lithium carbonate, sodium carbonate, potassium carbonate, magnesium carbonate, calcium carbonate and barium carbonate; phosphoric acid salts of alkali metals and/or alkaline earth metals such as lithium phosphate, sodium phosphate, potassium phosphate, magnesium phosphate, calcium phosphate and barium phosphate; and boric acid salts of alkali metals and/or alkaline earth metals such as lithium borate, sodium borate, potassium borate, magnesium borate, calcium borate, barium borate.

As specific examples of nitrogen-containing compounds there may be mentioned aliphatic amines such as ammonia, methylamine, dimethylamine, trimethylamine, ethylamine, diethylamine, triethylamine, ethanolamine, diethanolamine, triethanolamine, ethylenediamine, N,N-dimethylethylenediamine and N,N,N′,N′-tetramethylethylenediamine, heterocyclic amines such as pyridine, methylpyridine, bipyridine, hydropyridine and phenanthroline, and aromatic amines such as aniline, diphenylamine, triphenylamine and the like, either as gases or aqueous solutions.

As specific examples of phosphorus-containing compounds there may be mentioned trialkylphosphines such as trimethylphosphine and triethylphosphine, triarylphosphines such as triphenylphosphine and tris(2-methoxyphenyl)phosphine, monodentate phosphines including diarylalkylphosphines such as diphenylmethylphosphine and diphenylethylphosphine, bidentate phosphines such as 1,2-diphenylphosphinoethane and 1,4-bisdiphenylphosphinobutane, trialkyl phosphites such as trimethyl phosphite, triethyl phosphite and tributyl phosphite, and triaryl phosphites such as triphenyl phosphite.

These may be used alone or in appropriate combinations of two or more.

Preferred for use among those mentioned above are lithium acetate, sodium acetate, potassium acetate, lithium propionate, sodium propionate, potassium propionate, lithium acrylate, sodium acrylate, potassium acrylate, lithium methacrylate, sodium methacrylate, potassium methacrylate, lithium hydroxide, sodium hydroxide, potassium hydroxide, lithium carbonate, sodium carbonate, potassium carbonate, pyridine, phenanthroline, trimethylphosphine, triethylphosphine, triphenylphosphine and tris(2-methoxyphenyl)phosphine. Even more preferred are lithium acetate, sodium acetate, potassium acetate, lithium carbonate, sodium carbonate, potassium carbonate, trimethylphosphine and triphenylphosphine.

The total amount of addition of the one or more compounds selected from the group consisting of basic compounds, nitrogen-containing compounds and phosphorus-containing compounds will depend on the type of starting material used, but it is preferably in the range of 0.001-3 mol, more preferably 0.01-2 mol, even more preferably 0.05-1.8 mol and most preferably 0.1-1.5 mol with respect to 1 mol of the compound represented by general formula (1). Addition with an amount in this range is preferred from the viewpoint of yield and economy.

The compounds selected from among basic compounds, nitrogen-containing compounds and phosphorus-containing compounds may be loaded on the catalyst during catalyst production, or they may be added to the reaction system either before or during reaction after catalyst production.

A solvent is not necessary for the acyloxy compound production process of the invention, but an organic solvent may optionally be used. As examples of organic solvents there may be mentioned aromatic hydrocarbons such as benzene; aliphatic hydrocarbons such as pentane, hexane, cyclohexane and heptane; ethers such as diethyl ether and diisopropyl ether; halogenated hydrocarbons such as chloroform, methylene chloride, dichloroethane and chlorobenzene; and carboxylic acid esters such as methyl acetate, ethyl acetate, methyl propionate, ethyl propionate, methyl (meth)acrylate and ethyl (meth)acrylate.

The amount of organic solvent used will depend on the starting materials used, but it may be in the range of 0-200 mass %, preferably 0-100 mass %, even more preferably 0-80 mass % and most preferably 0-70 mass % of the total starting material weight. Using an amount in this range is preferred from the viewpoint of yield and economy.

When the compound represented by general formula (1) and the obtained acyloxylation product used in the acyloxy compound production process of the invention are polymerizable compounds, it is preferred to conduct the reaction in the presence of a polymerization inhibitor in order to inhibit polymerization of the compounds and enhance the yield. As polymerization inhibitors there may be mentioned quinone-based polymerization inhibitors such as hydroquinone, methoxyhydroquinone, benzoquinone and p-tert-butylcatechol; alkylphenol-based polymerization inhibitors such as 2,6-di-tert-butylphenol, 2,4-di-tert-butylphenol, 2-tert-butyl-4,6-dimethylphenol, 2,6-di-tert-butyl-4-methylphenol and 2,4,6-tri-tert-butylphenol; amine-based polymerization inhibitors such as alkylated diphenylamine, N,N′-diphenyl-p-phenylenediamine and phenothiazine; and copper dithiocarbamate-based polymerization inhibitors such as: copper dimethyldithiocarbamate, copper diethyldithiocarbamate and copper dibutyldithiocarbamate. These may also be used alone or in appropriate combinations of two or more. Preferred for use among the above are quinone-based polymerization inhibitors, and especially hydroquinone, methoxyhydroquinone, benzoquinone, p-tert-butylcatechol and phenothiazine.

The amount of polymerization inhibitor added will depend on the type of compound represented by general formula (1) that is used, but it is preferably an amount in the range of 0.001-5 mass %, more preferably 0.005-1 mass % and most preferably 0.01-0.1 mass % of the polymerizable compound. Addition with an amount in this range is preferred from the viewpoint of polymerization inhibition and yield.

The reaction temperature is preferably in the range of 0-500° C. and most preferably in the range of 30-300° C. The reaction time may be appropriately set according to the types, combination and amounts of starting materials, catalyst and organic solvent, in order to bring the reaction to completion. The reaction pressure will depend on the starting materials and the reaction temperature, and may be ordinary pressure (atmospheric pressure) or pressurization. Examples of reaction systems include batch systems, semi-batch systems and continuous systems.

The acyloxylation product obtained by the process of the invention may be obtained by separation of the catalyst and purification of the reaction solution. The purification means is not particularly restricted and may be one that accomplishes separation and purification by distillation, extraction, column chromatography or the like. These methods may also be used in combination. The most preferred methods among these are distillation and extraction.

The starting materials and organic solvent that are separated by the purification step may also be reused in the reaction. The separated catalyst may likewise be reused in the reaction.

The present invention will now be explained in greater detail by examples, with the understanding that the invention is in no way restricted by the examples.

### Production of Acyloxylation Catalysts

### Example 1

### Production of Catalyst A-1

A silica spherical support (sphere diameter: 5 mm, area-to-weight ratio: 160 m²/g, absorption percentage: 0.75 g/g, HSV-I by Shanghai Kaigen) was used to produce catalyst A-1 by the following procedure.

Step 1. A 23 g portion of the support (moisture absorption: 19.7 g) was impregnated with an aqueous solution containing 1.5 g of a 56 mass % Na₂PdCl₄ aqueous solution, 1.5 g of a 17 mass % HAuCl₄ aqueous solution and 0.7 g of a 20 mass % FeCl₃.6H₂O aqueous solution, in an amount equivalent to the moisture absorption of the support, to obtain a catalyst precursor.

Step 2. The support/catalyst precursor obtained in step 1 was immersed in an aqueous solution containing 3 g of Na₂SiO₃.9H₂O and allowed to stand at room temperature for 20 hours.

Step 3. To the aqueous solution of step 2 there was added 4 ml of a 53 mass % hydrazine hydrate aqueous solution, and after gentle mixing, the mixture was allowed to stand at room temperature for 4 hours. The reduced catalyst precursor was washed with running water until disappearance of chloride ion. The washed catalyst precursor was then dried at approximately 110° C. for 4 hours.

Step 4. the catalyst precursor obtained in step 3 was immersed in 2 L of a 1 mass % sulfuric acid aqueous solution. It was then washed overnight with running water and dried at 110° C. for 4 hours.

Step 5. the acid treated catalyst precursor obtained in step 4 was impregnated with an aqueous solution containing 2 g of potassium acetate, in an amount equivalent to the moisture absorption of the support, and dried at 110° C. for 4 hours.

The measured precipitation-starting pH values of the Na₂PdCl₄ aqueous solution, HAuCl₄ aqueous solution and FeCl₃.6H₂O aqueous solution were 4.5, 4.0 and 2.5, respectively.

### Example 2

### Production of Catalyst A-2

Catalyst A-2 was produced by the same procedure as in Example 1, except that step 4 was omitted.

### Example 3

### Production of Catalyst B-1

Catalyst B-1 was produced by the same procedure as in Example 1, except that the FeCl₃.6H₂O aqueous solution used in step 1 was changed to a TiCl₄ aqueous solution. The measured precipitation-starting pH of the TiCl₄ aqueous solution was 1.5.

### Example 4

### Production of Catalyst B-2

Catalyst B-2 was produced by the same procedure as in Example 3, except that step 4 was omitted.

### Example 5

### Production of Catalyst C-1

Catalyst C-1 was produced by the same procedure as in Example 1, except that the FeCl₃.6H₂O aqueous solution used in step 1 was changed to a ScCl₃.6H₂O aqueous solution and the 2 L of the 1 mass % sulfuric acid aqueous solution in step 4 was changed to 200 ml of a 1 mass % phosphoric acid aqueous solution. The measured precipitation-starting pH of the ScCl₃.6H₂O aqueous solution was 4.0.

### Example 6

### Production of Catalyst G-1

Catalyst G-1 was produced by the same procedure as in Example 2, except that the amount of HAuCl₄ aqueous solution added in step 1 was changed to 3.0 g and the amount of FeCl₃.6H₂O aqueous solution added was changed to 1.4 g.

### Comparative Example 1

### Production of Catalyst D-1

Catalyst D-1 was produced by the same procedure as in Example 2, except that the FeCl₃.6H₂O aqueous solution used in step 1 was not added.

### Comparative Example 2

### Production of Catalyst E-1

Catalyst E-1 was produced by the same procedure as in Example 1, except that the FeCl₃.6H₂O aqueous solution used in step 1 was changed to a ZnCl₂ aqueous solution. The measured precipitation-starting pH of the ZnCl₂ aqueous solution was 7.0.

### Comparative Example 3

### Production of Catalyst E-2

Catalyst E-2 was produced by the same procedure as in Comparative Example 2, except that step 4 was omitted.

### Comparative Example 4

### Production of Catalyst F-1

Catalyst F-1 was produced by the same procedure as in Example 2, except that the FeCl₃.6H₂O aqueous solution used in step 1 was changed to a BaCl₂.2H₂O aqueous solution. The measured precipitation-starting pH of the BaCl₂.2H₂O aqueous solution was 11.0.

### Catalyst Evaluation

Reaction with each of the obtained catalysts was evaluated by the following method.

### Catalytic Activity Evaluation

After diluting 3 cc of the catalyst with 75 cc of glass beads, it was packed into a reaction tube (SUS316 L, inner diameter: 22 mm, length: 480 mm). Reaction was conducted with a reaction temperature of 150° C., a reaction pressure of 0.6 MPaG and circulation of gas with the composition C₂H₄/O₂/H₂O/HOAc/N₂=47.3/6.1/5.6/26.3/14.7 (mol %) at 20 nL/h.

The product gas and product liquid were sampled between 2 h and 4 h after start of the reaction, as 4 h reaction samples. The product gas and product liquid were also sampled between 96 h and 98 h after start of the reaction, as 98 h reaction samples. The following analysis was then performed and the activity and selectivity for vinyl acetate product were calculated.

Analysis of the reactor exit gas was carried out by the following method.

### 1. Oxygen

Using an absolute calibration curve method, 50 ml of efflux gas was sampled and the total amount was directed into the 1 ml gas sampler of a gas chromatograph for analysis under the following conditions.

Gas chromatograph: Shimadzu gas chromatography gas sampler (MGS-4: 1 ml metering tube)-equipped gas chromatograph (GC-14 (B) by Shimadzu Corp.)
Column: MS-5A IS 60/80 mesh (3 mmΦ×3 m)
Carrier gas: helium (flow rate: 20 ml/min)
Temperature conditions: Detector temperature and vaporizing chamber temperature=110° C., column temperature=70° C., fixed.
Detector: TCD (He pressure: 70 kPaG, Current: 100 m (A))

### 2. Acetic Acid

Using an internal standard method, 1 ml of 1,4-dioxane was added as the internal standard to 10 ml of reaction solution to prepare a solution for analysis, and 0.2 μl thereof was injected and analyzed under the following conditions.

Gas chromatograph: GC-14B by Shimadzu Corp.
Column: Thermon 3000 packed column (length: 3 m, inner diameter: 0.3 mm)
Carrier gas: Nitrogen (flow rate: 20 ml/min)
Temperature conditions: Detector temperature and vaporizing chamber temperature: 180° C., column temperature: 50° C. maintained for 6 minutes from start of analysis, increased to 150° C. thereafter at a temperature-elevating rate of 10° C./min, and held at 150° C. for 10 minutes.
Detector: FID (H₂ pressure: 40 kPaG, air pressure: 100 kPaG)

### 3. Vinyl Acetate

Using an internal standard method, 1 g of n-propyl acetate was added as the internal standard to 6 g of reaction solution to prepare a solution for analysis, and 0.3 μl thereof was injected and analyzed under the following conditions.

Gas chromatograph: GC-9A by Shimadzu Corp.
Column: TC-WAX capillary column (length: 30 m, inner diameter: 0.25 mm, film thickness: 0.5 μm)
Carrier gas: Nitrogen (flow rate: 30 ml/min)
Temperature conditions: Detector temperature and vaporizing chamber temperature: 200° C., column temperature: 45° C. maintained for 2 minutes from start of analysis, increased to 130° C. thereafter at a temperature-elevating rate of 4° C./min, held at 130° C. for 15 minutes, increased to 200° C. thereafter at a temperature-elevating rate of 25° C./min, and held at 200° C. for 10 minutes.
Detector: FID (H₂ pressure: 60 kPaG, air pressure: 100 kPaG)

The results of reaction evaluation for each of the catalysts of Examples 1-6 and Comparative Examples 1-4 are shown in Table 1.

The values for 4 h activity in the table are expressed as the activity calculated for each catalyst based on the amount of vinyl acetate produced upon 4 h of reaction under the conditions described above, divided by the activity of Comparative Example D-1.

The selectivity is expressed as the selectivity for vinyl acetate calculated by analysis after 4 h of reaction. The activity reduction is expressed as the value of the activity after 98 h of reaction, divided by the activity after 4 h of reaction, as an index of the reduction in activity.

The selectivity and activity were defined as follows.

Selectivity (%)=(amount of vinyl acetate (mol) in total after reaction/total (mol) after reaction)

Activity (g/L/h)=(amount of vinyl acetate produced per unit time (g/h)/volume of catalyst (L))

The loading weights for each of the elements in the production of each catalyst (Examples 1-6 and Comparative Examples 1-4) are shown in Table 2. In Table 2, the values for Examples 1, 3 and 5 and Comparative Example 2 are those prior to acid washing.

TABLE 1

| | | | | | | | Non- | | | | | |
|---|---|---|---|---|---|---|---|---|---|---|---|---|
| | | | | | | | solubilizing | | Acid | 4 h | | Activity |
| | | (a) | (b) | (c) | (e) | Other | treatment | Reduction | treatment | activity | Selectivity | reduction |
| | Catalyst | Element | | | | | +(performed)/−(not performed) | | | /H-3 | % | 4 h/98 h |
| Ex. 1 | A-1 | Pd | Au | Fe | K | — | + | + | + | 1.21 | 88.9 | |
| Ex. 2 | A-2 | Pd | Au | Fe | K | — | + | + | − | 1.27 | 88.7 | 0.84 |
| Ex. 3 | B-1 | Pd | Au | Ti | K | — | + | + | + | 1.17 | 89.3 | |
| Ex. 4 | B-2 | Pd | Au | Ti | K | — | + | + | − | 1.13 | 89.4 | |
| Ex. 5 | C-1 | Pd | Au | Sc | K | — | + | + | + | 1.05 | 89.9 | |
| Ex. 6 | G-1 | Pd | Au | Fe | K | — | + | + | − | 1.35 | 89.3 | 0.86 |
| Comp. | D-1 | Pd | Au | — | K | — | + | + | − | 1.00 | 89.3 | 0.78 |
| Ex. 1 | | | | | | | | | | | | |
| Comp. | E-1 | Pd | Au | — | K | Zn | + | + | + | 1.08 | 89.3 | |
| Ex. 2 | | | | | | | | | | | | |
| Comp. | E-2 | Pd | Au | — | K | Zn | + | + | − | 1.03 | 89.1 | |
| Ex. 3 | | | | | | | | | | | | |
| Comp. | F-1 | Pd | Au | — | K, Ba | — | + | + | − | 1.29 | 90.9 | 0.64 |
| Ex. 4 | | | | | | | | | | | | |

TABLE 2
(<mass %>)

| | Pd | Au | Fe | Ti | Sc | K | Ba | Zn |
|---|---|---|---|---|---|---|---|---|
| Example 1 | 1.2 | 0.6 | 0.17 | | | 3.2 | | |
| Example 2 | 1.2 | 0.6 | 0.17 | | | 3.2 | | |
| Example 3 | 1.2 | 0.6 | | 0.15 | | 3.2 | | |
| Example 4 | 1.2 | 0.6 | | 0.15 | | 3.2 | | |
| Example 5 | 1.2 | 0.6 | | | 0.14 | 3.2 | | |
| Example 6 | 1.2 | 1.2 | 0.34 | | | 3.2 | | |
| Comparative | 1.2 | 0.6 | | | | 3.2 | | |
| Example 1 | | | | | | | | |
| Comparative | 1.2 | 0.6 | | | | 3.2 | | 0.20 |
| Example 2 | | | | | | | | |
| Comparative | 1.2 | 0.6 | | | | 3.2 | | 0.20 |
| Example 3 | | | | | | | | |
| Comparative | 1.2 | 0.6 | | | | 3.2 | 4.4 | |
| Example 4 | | | | | | | | |

### INDUSTRIAL APPLICABILITY

The present invention can provide an acyloxylation catalyst with excellent balance of performance between initial reaction activity, selectivity and sustained activity, and it is therefore industrially useful.

1. A process for production of an acyloxylation catalyst comprising a step of loading (a) a first component containing at least one element of Groups 8, 9, 10 and 11 of the Periodic Table, (b) a second component containing an element which is at least one element of Groups 8, 9, 10 and 11 of the Periodic Table and which is different from the element of the first component and (c) a third component containing an element which is a component that has a precipitation-starting pH below the precipitation-starting pH of the first component and second component and which is different from the elements of the first component and second component, together, onto (d) a support.

2. A process for production of an acyloxylation catalyst according to claim 1, wherein the step of loading onto the (d) support is followed by non-solubilizing treatment.

3. A process for production of an acyloxylation catalyst according to claim 1, wherein the step of loading onto the (d) support is followed by reduction treatment.

4. A process for production of an acyloxylation catalyst according to claim 3, wherein the reduction treatment is followed by contact with an acid and/or chelating agent.

5. A process for production of an acyloxylation catalyst according to claim 1, comprising further (e) adding a fourth component containing at least one element of Groups 1 and 2 of the Periodic Table except for hydrogen.

6. A process for production of an acyloxylation catalyst according to claim 1, wherein the (a) first component includes palladium.

7. A process for production of an acyloxylation catalyst according to claim 1, wherein the (b) second component includes an element of Group 11 of the Periodic Table.

8. A process for production of an acyloxylation catalyst according to claim 7, wherein the (b) second component includes at least one element selected from the group consisting of gold and copper.

9. A process for production of an acyloxylation catalyst according to claim 1, wherein the (c) third component includes an element of Groups 3-13 of the Periodic Table.

10. A process for production of an acyloxylation catalyst according to claim 1, wherein the proportion of the loading weight of the element of the (b) second component to the loading weight of the element of the (a) first component is 0.4-1.5.

11. A process for production of an acyloxylation catalyst according to claim 1, wherein the proportion of the loading weight of the element of the (c) third component to the loading weight of the element of the (b) second component is 0.1-0.5.

12. An acyloxylation catalyst obtained by a process according to claim 1.

13. A process for production of acyloxy compounds, comprising reacting, in the presence of a catalyst obtained by a process according to claim 1, a compound represented by the general formula (1): CHR¹R²—X (wherein R¹ and R² each independently represent hydrogen or an organic residue and X represents an optionally substituted aromatic hydrocarbon residue or optionally substituted olefin residue) or ethylene, with a carboxylic acid represented by the general formula (2): R³—COOH (wherein R³ represents hydrogen or an organic residue) and oxygen to produce a compound represented by the general formula (3): R³—COO—CR¹R²—X (wherein R¹, R², R³ and X have the same definitions as above) or vinyl acetate.

14. A process for production of an acyloxy compound according to claim 13, wherein the reaction is conducted also in the presence of at least one compound selected from the group consisting of basic compounds, nitrogen-containing compounds and phosphorus-containing compounds.
